(19) United States
(12) Patent Application Publication
(10) Pub. No.: US 2026/0363950 A1
(43) Pub. Date: Nov. 19, 2026

(54) CONTAINER SYSTEMS AND METHODS FOR CRYOGENIC STORGE AND DELIVERY OF BIOINK

(71) Applicant: West Pharmaceutical Services, Inc., Exton, PA (US)

(72) Inventors: Alexander LYNESS, West Chester, PA (US); Didarul Bahar BHUIYAN, York, PA (US); Anupama PRABHATHACHANDRAN, Malvern, PA (US); Lauren NUTT, Exton, PA (US); Linda TREBING, Wayne, PA (US); Eleftherios HRISTOFAS, Lancaster, PA (US); Md Abu HASAN, Exton, PA (US); Anthony J. WIRTEL, III, Malvern, PA (US); Michael SULLIVAN, Birdsboro, PA (US); Christian EICHHORN, Pottstown, PA (US); Bryan F. ALLARD, Royersford, PA (US); William B. MATAKAS, Jr., Exton, PA (US)

(21) Appl. No.: 19/155,534

(22) PCT Filed: Feb. 9, 2024
(86) PCT No.: PCT/US2024/015159

§ 371 Date: Aug. 11, 2025

Related U.S. Application Data
(60) Provisional application No. 63/444,355, filed on Feb. 9, 2023.
(60) Provisional application No. 63/449,166, filed on Mar. 1, 2023.

(51) Int. Cl. A61J 1/05; A61J 1/14; A61M 5/31; B33Y 30/00

(52) U.S. Cl. CPC A61J 1/05; A61J 1/1406; A61J 1/1412; A61J 1/1443; A61M 5/3135; B33Y 30/00; A61M 2005/3104; A61M 2005/3121

(57) ABSTRACT

Container systems including a syringe for containing bioink and for providing container closure integrity through cryogenic storage and warming are described. The syringe includes a barrel and a stopper that seals a proximal opening of the syringe. The syringe also includes a cap that is removably connected to a connector of the syringe at a distal end of the syringe and that seals a distal opening of the syringe. The syringe includes a piston within the barrel that can be spaced apart from the stopper when the barrel is filled with the bioink. The stopper and the cap can respectively seal the proximal and distal opening throughout a temperature range of between −196 °C and 37 °C.

### CROSS REFERENCE TO RELATED APPLICATIONS

This application claims priority to U.S. Provisional Patent App. No. 63/444,355, filed on Feb. 9, 2023 and to U.S. Provisional Patent App. No. 63/449,166, filed on Mar. 1, 2023. The entirety of each priority application (i.e., U.S. Provisional Patent App. Nos. 63/444,355 and 63/449,166) are hereby incorporated herein by reference.

### FIELD OF THE INVENTION

The present disclosure generally relates to primary container systems and methods, and more particularly, to syringes, caps, and assemblies that can store a bioink, maintain container closure integrity between cryogenic and room temperatures, and that can connect to a bioprinter for delivery of the bioink.

### BACKGROUND

Bioprinting is an extended application of additive manufacturing where bioinks are deposited, layer-by-layer, for fabrication of functional tissues and organs. Bioprinting can be used for regenerative medicine to, for example, address organ shortage challenges, for pharmaceutical testing, for food production, among other possibilities.

Various techniques exist for the deposition of bioinks including pneumatic extrusion, ink-jet printing, extrusion printing, stereolithography, laser assisted bioprinting techniques, among others. For example, pneumatic drivers with compressed air at pressures ranging from 10-200 kPa can be used for pneumatic extrusion bioprinters.

Bioinks used in current bioprinters are often stored in refrigerators at relatively warm temperatures (e.g., 4 °C). As a result, the bioinks can have short shelf life ranging from a few weeks to up to a few months. The short shelf life of bioinks can limit the growth of bioprinting. Cryogenic storage of bioinks could improve longevity, but cryogenic storage has not been widely implemented at least in part because current ready-to-use containers for bioinks cannot maintain container closure integrity throughout a freezing and thawing cycle.

There thus exists a need for primary container systems for bioprinting that can maintain container closure integrity during cryogenic storage and thawing. There is a need for the primary container systems to also be adaptable to the delivery of bioink. There is a need for the primary container systems to also be capable of being sterilized and to maintain sterility throughout the process of filling, freezing, thawing and delivery of bioinks.

Some substances, such as medicines or vaccines, require storage at low temperatures to prevent degradation and/or maintain efficacy for the intended use of the substances. Container closure integrity (CCI) of storage containers that hold such substances should be maintained at these low temperatures to prevent contamination, degradation, or other undesirable changes.

Vials provide one solution for storing such substances at low temperatures while preserving CCI. But transferring the substances out of vials can be complex, costly, and can risk contamination or unintended needle sticks during substance transfer.

Insert needle syringes and current Luer lock syringes can reduce the complexity and some risks associated with the vials. But insert needle syringes and current Luer lock syringes do not reliably maintain CCI in low temperature environments that such substances will be storage at. For example, materials of the insert needle syringes may degrade at low temperatures, which may compromise CCI, clog the needle, or contaminate the substances. Caps that can provide CCI on the distal end of current Luer lock syringes do not maintain CCI at low temperatures that these substances will be stored at. For example, current caps can contract at different rates relative to the distal end of the Luer lock syringe, which can create gaps that compromise CCI at low temperatures.

Accordingly, there exists a need for syringe solutions that enable storage of substances within the syringes at low temperatures while maintaining CCI at the distal end of the syringes.

### SUMMARY

The foregoing needs are met by the primary container systems, caps, and methods of the present disclosure. According to one aspect of the present disclosure, a syringe configured to contain a bioink and to provide container closure integrity through cryogenic storage and warming. The syringe also includes a barrel with a proximal end defining a proximal opening and a distal end defining a distal opening. The distal end may include a connector. The syringe also includes a stopper at the proximal end, and the stopper seals the proximal opening. The syringe also includes a cap removably connected to the connector and the cap seals the distal opening. The syringe also includes a piston within the barrel and the piston is configured to be spaced apart from the stopper when the barrel is filled with the bioink. The stopper is configured to seal the proximal opening throughout a temperature range. The cap is configured to seal the distal opening throughout the temperature range. The temperature range is between −196 °C and 37 °C.

Implementations may include one or more of the following features. The temperature range includes all temperatures between −196 °C and 37 °C. The syringe may include a filter within the barrel between the proximal opening and the piston. The syringe may include a ledge that the filter is connected to. The insert is connected to an inner wall of the barrel. During cryogenic storge the piston is spaced distally from the filter. The filter has a pore size of between 0.2 and 0.3 μm. The connector is a Luer lock connector. The proximal end may include a flange, and the syringe may include a crimp that crimps the stopper to the flange. The barrel may include a polymer. The polymer is a cyclic olefin polymer. The piston may include an elastomer. The stopper may include an elastomer. A central portion of the stopper is pierceable. The bioink fills the barrel between the piston and the cap. The bioink may include at least one of living cells or extracellular matrix components. The bioink occupies a first volume within the barrel when the syringe and the bioink are at room temperature and the bioink occupies a second volume within the barrel when the syringe and the bioink are at a cryogenic storge temperature, and the second volume is greater than the first volume. The connector of the distal end of the barrel is a first connector. The first connector may include a first material having a first coefficient of linear thermal expansion. The cap may include a second connector removably connected to the first connector. The second connector may include a second material having a second coefficient of linear thermal expansion, and a seal configured to engage with the distal opening of the barrel. The second coefficient of linear thermal expansion is less than or equal to the first coefficient of linear thermal expansion. The second coefficient of linear thermal expansion is within 10% of the first coefficient of linear thermal expansion. The distal end of the barrel may include a nozzle that defines the distal opening, and the body and the nozzle impart a compressive force on the seal. The seal conforms to the distal end of the barrel or occludes the distal opening of the barrel at room temperature. The seal may include a proximal face that is engaged with the distal end of the barrel, and the cap may include an inert film that lines the proximal face. The seal may include an elastomer, the elastomer of the seal has a third coefficient of linear thermal expansion, and the third coefficient of linear thermal expansion is within 10% of the first coefficient of linear thermal expansion. The seal is engaged with at least one of the distal opening of the barrel or exterior walls of the distal end of the barrel. The distal end of the barrel may include the first material. The seal may include a third material having a third coefficient of linear thermal expansion, and the third coefficient of linear thermal expansion is greater than the first coefficient of linear thermal expansion. The cap may include: a body defining a recess; a connector removably connected to the connector of the barrel; and a seal engaged with the distal opening. The seal may include a first material and the body may include a second material, and the first material and the second material are different. The seal is within a distal end of the recess. The seal may include a proximal face that seals the distal opening of the barrel, and the proximal face is lined with an inert film. The seal has a glass transition temperature of −80 °C or lower. The spring imparts a compressive force on the seal to seal the distal opening of the barrel. The material has a negative coefficient of thermal expansion and imparts a compressive force on the seal to seal the distal opening of the barrel when the material is cooled. The seal has a coefficient of linear thermal expansion at temperatures below a glass transition temperature of the seal that is substantially equivalent to a coefficient of linear thermal expansion of cyclic olefin polymer. The cap may include: a connector removably connected to the connector of the distal end of the barrel; and a body connected to the connector of the cap and engaged with the distal opening of the barrel. The connector of the cap may include a cyclic olefin polymer and the body may include an elastomer.

One general aspect includes an assembly for a bioprinter. The assembly includes a syringe. The syringe may include a barrel that may include a proximal end defining a proximal opening and a distal end defining a distal opening. The distal end may include a connector. The syringe may include a stopper at the proximal end, and the stopper may seal the proximal opening. The syringe may include a bioink within the barrel and a piston within the barrel between the stopper and the bioink. The syringe may include a filter within the barrel between stopper and the piston. The assembly also includes an adapter configured to connect to the proximal end of the barrel. The assembly also includes a needle extending through the adapter and configured to pierce both the stopper and the filter.

Implementations may include one or more of the following features. The needle is configured to pneumatically supply a gas within the barrel to move the piston distally and dispense the bioink from the distal end of the barrel. The adapter is configured to support the needle at a position in which a distal tip of the needle is within the barrel distal to the filter and proximal to the piston. The adapter may include a first body and a second body, and the needle is configured to extend through the first body. The first body may include a first connector and the second body may include a second connector that is complementary to the first connector. The first connector and the second connector are configured to connect to join the first body to the second body. The second body may include a fitting configured to fit the proximal end of the barrel to the adapter. The fitting may include a recess and an opening in the second body, and the recess is configured to receive the proximal end of the barrel and the stopper. The barrel is configured to extend through the opening. The assembly may include a nozzle that is configured to removably connect to the connector. The adapter is configured to operably connect the syringe to the bioprinter. The bioink may include at least one of living cells or extracellular matrix components.

One general aspect includes a syringe with a barrel that may include a nozzle and a first connector at a distal end of the barrel. The first connector may include a first material having a first coefficient of linear thermal expansion. The syringe also includes a cap that may include a body defining a recess configured to receive the nozzle, and a second connector configured to connect to the first connector. The second connector may include a second material having a second coefficient of linear thermal expansion. The cap also includes a seal configured to engage with the nozzle when the second connector is connected to the first connector. The second coefficient of linear thermal expansion is less than or equal to the first coefficient of linear thermal expansion.

Implementations may include one or more of the following features. The syringe where the second coefficient of linear thermal expansion is within 10% of the first coefficient of linear thermal expansion. The first connector is a first Luer lock connector and the second connector is a second Luer lock connector that is complimentary to the first Luer lock connector. The nozzle is a Luer cone tip. The seal is configured to engage with the nozzle when the second connector is connected to the first connector to form a liquid-tight seal with the nozzle to preserve container closure integrity at temperatures between −196 °C and −20 °C. The seal is configured to engage with the nozzle when the second connector is connected to the first connector to form an air-tight seal with the nozzle to preserve container closure integrity at temperatures between −196 °C and −20 °C. The body and the nozzle impart a compressive force on the seal when the second connector is connected to the first connector. The seal conforms to the nozzle or occludes an opening of the nozzle at room temperature when the second connector is connected to the first connector. The seal may include a proximal face that is configured to engage with the nozzle, and the cap may include an inert film that lines the proximal face. The inert film may include a fluorinated polymer material. The proximal face is substantially flat, concave, or convex. The seal may include an elastomer. The elastomer of the seal has a third coefficient of linear thermal expansion, and the third coefficient of linear thermal expansion is within 10% of the first coefficient of linear thermal expansion. The seal is configured to engage at least one of an opening of the nozzle or exterior walls of the nozzle. The nozzle may include the first material, and the seal may include a third material having a third coefficient of linear thermal expansion. The third coefficient of linear thermal expansion is greater than the first coefficient of linear thermal expansion.

One general aspect includes a cap configured to seal a nozzle of a barrel of a syringe. The cap includes a body defining a recess configured to receive the nozzle. The cap also includes a connector configured to connect to a connector of the barrel. The cap also includes a seal configured to engage with the nozzle when the connector of the cap is connected to the connector of the barrel. The seal may include a first material and the body may include a second material, and the first material and the second material are different.

Implementations may include one or more of the following features. The connector of the cap may include a cyclic olefin polymer and the first material is an elastomer. The seal is within a distal end of the recess and the seal may include a proximal face that is configured to seal the nozzle. The proximal face is lined with an inert film. The inert film may include a fluorinated polymer material. The seal has a glass transition temperature of −80 °C or lower. The spring is configured to impart a compressive force on the seal to seal the nozzle when the connector of the cap is connected to the connector of the barrel. The material has a negative coefficient of thermal expansion and is configured to impart a compressive force on the seal to seal the nozzle when the connector of the cap is connected to the connector of the barrel and when the material is cooled. The seal has a coefficient of linear thermal expansion at temperatures below a glass transition temperature of the seal that is substantially equivalent to a coefficient of linear thermal expansion of cyclic olefin polymer.

One general aspect includes a cap that is configured to seal a nozzle of a barrel of a syringe. The cap includes a connector configured to connect to a connector of the barrel. The cap also includes a body connected to the connector and configured to engage with the nozzle when the connector of the cap is connected to the connector of the barrel. The connector of the cap may include a cyclic olefin polymer and the body may include an elastomer.

Implementations may include one or more of the following features. The body may include a proximal face that is configured to seal the nozzle. The proximal face is lined with an inert film. The inert film may include a fluorinated polymer material. The connector is overmolded onto the body.

One general aspect includes a method of preparing a syringe for bioprinting. The method includes sterilizing the syringe. The method also includes filling the syringe with a bioink from a distal opening of the syringe. The method also includes connecting a cap to the distal opening to form a distal seal. The method also includes connecting a stopper to a proximal opening of the syringe to form a proximal seal. The method also includes cooling the syringe to a cryogenic storage temperature. The method also includes warming the syringe from the cryogenic storage temperature to a bio printing temperature. Container closure integrity of the proximal seal and the distal seal is maintained during the cooling and the warming. The method also includes operably connecting the syringe to a bioprinter.

Implementations may include one or more of the following features. The cryogenic storage temperature is between −196 °C and −130 °C. The bio printing temperature is between 0 °C and 30 °C. Operably connecting the syringe to a bioprinter may include piecing the stopper and a filter of the syringe with a needle after the warming of the syringe. Operably connecting the syringe to a bioprinter may include connecting a proximal end of the syringe to an adapter. The method may include connecting a nozzle to a distal end of the syringe. The bioink may include at least one of living cells or extracellular matrix components.

There has thus been outlined certain embodiments of the present disclosure in order that the detailed description thereof herein may be better understood, and in order that the present contribution to the art may be better appreciated. There are additional embodiments of the present disclosure that will be described below, and which form the subject matter of the claims appended hereto.

In this respect, before explaining at least one aspect of the present disclosure in detail, it is to be understood that the embodiments described herein are not limited in their application to the details of construction and to the arrangements of the components set forth in the following description or illustrated in the drawings. The primary container system and methods of this disclosure are capable of aspects in addition to those described, and of being practiced and carried out in various ways.

### BRIEF DESCRIPTION OF THE DRAWINGS

In order that the present disclosure may be readily understood, aspects are illustrated by way of examples in the accompanying drawings, in which like parts are referred to with like reference numerals throughout.

FIG. 1 shows a schematic cross section view of a syringe.

FIG. 2 shows a partial, schematic cross section view of a first embodiment of the syringe.

FIG. 3 shows a partial, schematic cross section view of a second embodiment of the syringe.

FIG. 4 shows a bioprinter.

FIG. 5 shows a magnified, schematic cross section view of an adapter operably connecting a syringe to the bioprinter of FIG. 4.

FIG. 6 shows an exploded, schematic cross section view of the adapter and the syringe of FIG. 5.

FIG. 7 shows a cross section view of the syringe with a first example of the cap.

FIG. 8 shows a cross-section view of a planar seal.

FIG. 9 shows a cross-section view of a concave seal.

FIG. 10 shows a cross-section view of a convex seal.

FIG. 11 shows a cross section view of the syringe with a second example of the cap.

FIG. 12 shows a cross section view of the syringe with the cap of FIG. 11 wrapped in a wrap.

FIG. 13 shows a cross section view of the syringe with a third example of the cap FIG. 14 shows a cross section view of the syringe with a fourth example of the cap.

FIG. 15 shows a cross section view of the syringe with a fifth example of the cap.

FIG. 16 shows a perspective view of another syringe with the cap.

FIG. 17 shows a process of preparing a syringe for bioprinting.

FIG. 18 shows a schematic cross section view of a syringe.

FIG. 19 shows a schematic cross section view of the syringe of FIG. 18 after sterilization.

FIG. 20 shows a schematic cross section view of the syringe of FIG. 19 filled with bioink.

FIG. 21 shows a schematic cross section view of the syringe of FIG. 20 sealed with a stopper.

FIG. 22 shows a schematic cross section view of the syringe of FIG. 21 cooled to a cryogenic temperature.

FIG. 23 shows a schematic cross section view of the syringe of FIG. 22 warmed from the cryogenic temperature to a bioprinting temperature.

FIG. 24 shows a schematic cross section view of the syringe of FIG. 23 pierced by a needle.

FIG. 25 shows a schematic cross section view of the syringe of FIG. 24 during bioprinting.

### DETAILED DESCRIPTION

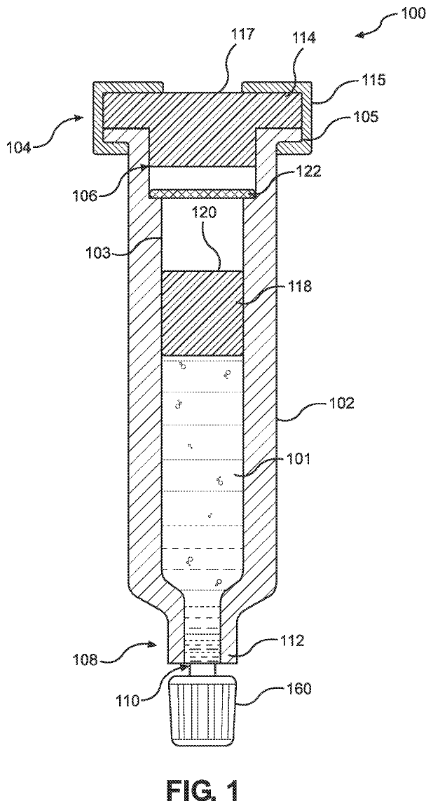

The present disclosure is directed to primary container systems and methods that can maintain container closure integrity during cryogenic storage and during thawing. The primary container systems and methods can be used for delivery of bioink in bioprinters. The primary container systems and methods can be sterilized and can maintain sterility throughout filling, freezing, thawing, and delivery of bioink. Such primary container systems and methods can eliminate the need to use different containers bioink storge, freezing and thawing, and/or printing, which can reduce system complexity, costs, and/or contamination risks. The primary container systems and methods can provide ready-to-use container system assembly that can integrate into existing commercial and research pneumatic and mechanical bioprinters. These and other aspects of the disclosure are described as follows in reference to FIGS. 1-15.

FIG. 1 shows a cross section view of a syringe 100 in accordance with aspects of the invention. The syringe 100 is an example of a primary container system of this invention. That is, the syringe 100 can maintain container closure integrity during storage and during thawing. The term “container closure integrity” as used herein can mean that the container (e.g., the syringe 100) is closed off from the outside environment so as not to exchange solids, liquids, and/or gases between an interior of the container and the outside environment. Maintaining container closure integrity during cryogenic storage and during thawing can include maintaining container closure integrity within a temperature range of between −196 °C and 37 °C. In embodiments, maintaining container closure integrity during cryogenic storage and during thawing can include maintaining container closure integrity at all temperatures between −196 °C and 37 °C. In embodiments, maintaining container closure integrity during cryogenic storage and during thawing can include at all temperatures between −196 °C and 37 °C.

The syringe 100 can include a barrel 102. The barrel 102 can contain the bioink 101. The term “bioink” as used herein can refer tissue structures, living cells, extracellular matrix components, biomaterials, hydrogels, extracellular proteins, growth factors, other bioactive components, other non-bioactive components, combinations thereof, etc. Due the container closure integrity provided at cryogenic temperatures by the primary container systems and methods of this disclosure, the bioink 101 can include living cells during storage, which can ensure sterility of the bioink 101 since the living cells would not need to be added between storage and bioprinting. Put differently, the primary container systems and methods of this disclosure can provide the opportunity to pre-mix living cells with other components of the bioink 101 and fill and store in the cryogenic conditions for long periods and use in a true “ready-to-print” format, that can significantly reduce the possibilities of contamination and variation. Additionally, although bioinks are used as an example substance that can be stored in the primary container systems and methods of this disclosure alternative substances can also be stored in the primary container systems. As such, the primary container systems and methods are not limited to bioinks and bioprinting applications.

The barrel 102 can include a proximal end 104 that defines a proximal opening 106. The barrel 102 can include a distal end 108 that defines a distal opening 110. The distal opening 110 can include a connector 112. The barrel 102 can comprises a polymer, glass, or other materials. For example, in embodiments the barrel 102 can comprise a cyclic olefin polymer, a cyclic olefin copolymer, and/or a polypropylene.

The syringe 100 can include a stopper 114. The stopper 114 can be provided at the proximal end 104 and can seal the proximal opening 106. The stopper 114 can seal the proximal opening 106 using any number of techniques. For example, in embodiments such as shown in FIG. 1, the stopper 114 can be crimped to the proximal end 104 of the barrel 102 with a crimp 115 to seal the proximal opening 106. The crimp 115 can be crimped to a flange 105 of the syringe 100. The stopper 114 can be pierceable. For example, a central portion 117 of the stopper can be pierced. The stopper 114 can be formed of any number of materials. For example, the stopper 114 can comprise an elastomeric polymer, a fluoropolymer (e.g., PTFE, PCTFE, FEP), a cyclic olefin polymer, a cyclic olefin copolymer, and/or a polypropylene. The crimp 115 can comprise a ductile material such as for example a ductile metal (e.g., aluminum).

The syringe 100 can include a cap 160. The cap 160 can be removably connected to the connector 112. The cap 160 can seal the distal opening 110. For example, the cap 160 and the connector 112 can each include respective complementary Luer lock connections to provide the removable connection between the cap 160 and the connector 112. Other connection types are possible including other threaded fittings, snap-fits, energized spring fits, etc. The stopper 114 and the cap 160 can respectively seal the proximal opening 106 and the distal opening 110 to maintain container closure integrity at each of the proximal opening 106 and the distal opening 110. The stopper 114 and the cap 160 can maintain the container closure integrity at each of the proximal opening 106 and the distal opening 110 during cryogenic storage and during thawing. As explained previously, this can include maintaining the container closure integrity at each of the proximal opening 106 and the distal opening 110 throughout a temperature range between −196 °C and 37 °C. In embodiments, the temperature range can at least include all temperatures between −196 °C and 37 °C. In embodiments, the temperature range can include all temperatures between −196 °C and 37 °C and at least some temperatures below −196 °C and/or above 37 °C. For example, the temperature range can include all temperatures between −196 °C and 37 °C. The cap 160 can comprise any number of materials that can maintain the seal of the distal opening 110. In embodiments, the cap 160 can comprise the same material as the material that forms the connector 112. For example, the connector 112 can be formed of a polymer (e.g., a cyclic olefin polymer) and the cap 160 can be formed of the same polymer (e.g., a cyclic olefin polymer). Since the connector 112 and the cap 160 can be formed of some or all of the same materials, the connector 112 and the cap 160 can expand and/or contract at similar rates to preserve container closure integrity at the distal opening 110. In embodiments, the cap 160 can include an o-ring between the distal opening 110 and the cap 160. The cap 160 can include a cap insert (not shown) that is adjacent to the distal opening 110 when the cap 160 is sealed against the connector 112. The o-ring and the cap insert can be comprised of an elastomer or functionally equivalent material.

The syringe 100 can include a piston 118. The piston 118 can be provided within the barrel 102. As discussed further herein, pressure can be applied (e.g., pneumatically) through the proximal opening 106 to a proximal surface 120 of the piston 118 to move the piston 118 distally and eject bioink from the syringe 100. The piston 118 can be formed of any number of materials. For example, the piston 118 can comprise an elastomeric polymer, a fluoropolymer (e.g., PTFE, PCTFE, FEP), a cyclic olefin polymer, a cyclic olefin copolymer, and/or a polypropylene.

The syringe 100 can include a filter 122. The filter 122 can be provided within the barrel 102 between the proximal opening 106 and the proximal surface 120 of the piston 118. The filter 122 can reduce or prevent contamination distal to the filter 122 within the interior of the barrel 102. In embodiments, the filter 122 can have a pore size of between 0.2 and 0.3 μm. For example, the filter 122 can have a pore size of 0.22 μm.

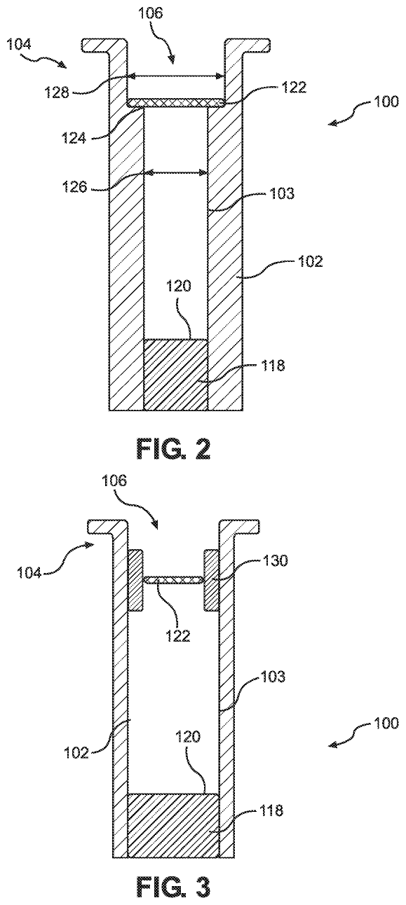

FIG. 2 shows a partial cross section view of the syringe 100 with a first embodiment of the filter 122. In embodiments such as shown in FIG. 2, the barrel 102 can include a ledge 124 and the filter 122 can be directly fixed to the ledge 124 using for example an adhesive or ultrasonic welding. The ledge 124 can partially or entirely circumferentially surround the barrel 102. In embodiments such as shown in FIG. 2, the barrel 102 can include a first internal diameter 126 and a second internal diameter 128 that is greater than the first internal diameter 126 and the ledge 124 can be step between the first internal diameter 126 and the second internal diameter 128.

FIG. 3 shows a partial cross section view of the syringe 100 with a second embodiment of the filter 122. In embodiments such as shown in FIG. 3, the syringe 100 can include an insert 130 that supports the filter 122 within the barrel. The insert 130 can be directly fixed to an inner wall 103 of the barrel 102 via any number of known techniques such as with adhesives or ultrasonic welding. In embodiments such as shown in FIG. 3, the barrel 102 can have a constant internal diameter from the proximal end 104 to the distal end 108. All syringe 100 embodiments disclosed herein can be provided with either the first embodiment of the filter 122 (FIG. 2) or the second embodiment of the filter 122 (FIG. 3).

When the barrel 102 is filled with a volume of the bioink the piston 118, i.e., the proximal surface 120 of the piston 118, can be spaced proximally a distance from the filter 122. The distance can be associated with a volume of the bioink 101, a material property of the bioink 101, and the range of temperatures the syringe 100 is configured to maintain container closure integrity across. For example, when the barrel 102 is filled with a volume of the bioink 101 and the syringe 100 is configured to maintain container closure integrity at cryogenic storage temperatures (e.g., temperatures below −130 °C, between −196 °C and −130 °C, below −196 °C), the distance can be greater than an amount of linear expansion (i.e., along the longitudinal axis of syringe) of the bioink 101 within the barrel 102 that results from cooling the bioink from a filling temperature to the cryogenic temperature. For example, in embodiments the bioink 101 can expand 9% or less. Since the distance is greater than the amount of linear expansion of the bioink 101, the bioink 101 can freely expand within the syringe 100 and move the piston 118 proximally towards the filter 122 without touching the filter 122 and thus without compromising the functionality of the filter 122. The filter 122 can allow gas (e.g., air) within the barrel 102 to flow through as the piston 118 moves proximally. Put differently, the bioink 101 can occupy a first volume within the barrel 102 when the syringe 100 and the bioink 101 are at a first temperature, or a filling temperature, which can be greater than 0 °C and in embodiments can be room temperature (i.e., between 20-22 °C). The bioink 101 can occupy a second volume within the barrel 102 when the syringe 100 and the bioink 101 are at a second temperature, i.e., a cryogenic storage temperature (e.g., a temperature below −130 °C, between −196 °C and −130 °C, below −196 °C). The second volume can be greater than the first volume and the proximal surface 120 can be spaced the distance from the filter 122 to accommodate the additional second volume without displacing the piston proximally as far as the filter 122.

The syringe 100 can be sterilized prior to filing with the bioink 101. The syringe 100 can be sterilized using any number of sterilization techniques including for example, gamma irradiation, steam autoclave, ethylene oxide, supercritical CO₂, etc. Different parts of the syringe 100 (e.g., the barrel 102, the piston 118, the filter 122, etc.) can be sterilized separately and assembled in a sterile manner. The entirety of the inner wall 103 of the barrel 102 can be sterilized. The filter 122 can maintain the sterility within the syringe 100 distally from the filter 122 by preventing contaminants that may enter from the proximal opening 106 when the syringe 100 is prepared for bioprinting.

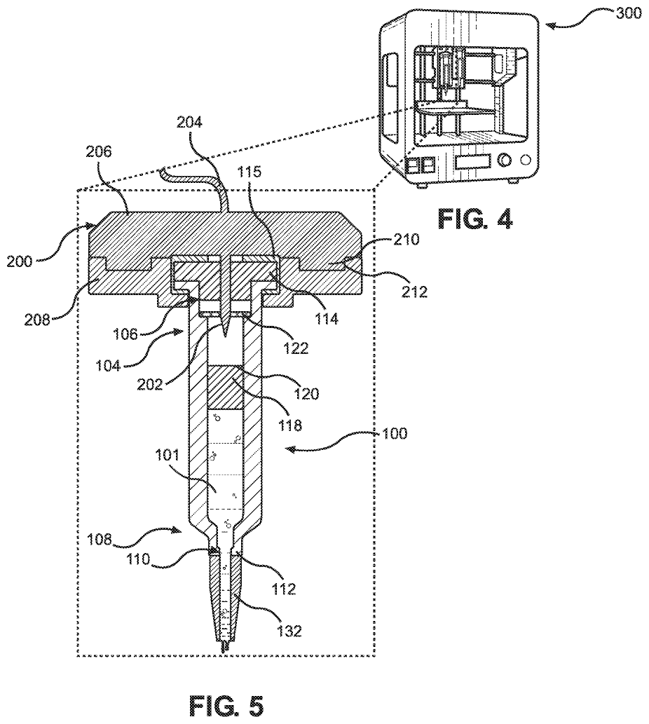

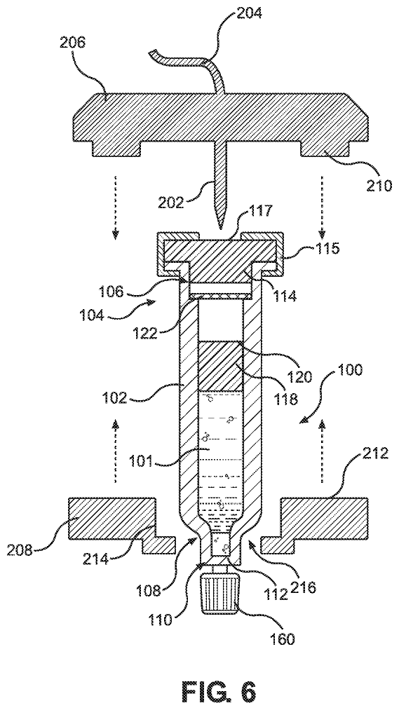

FIG. 4 shows a bioprinter 300. FIG. 5 shows a magnified, schematic cross section view of an adapter 200 operably connecting the syringe to the bioprinter 300. FIG. 6 shows an exploded, schematic cross section view of the adapter and the syringe of FIG. 5. The adapter 200 can operably connect any of previously-described embodiments of the syringe 100 to a bioprinter 300. The primary container systems can also include a nozzle 132. In embodiments, the nozzle 132 can be removably connected to the connector 112 in the same manner as the cap 160, as previously described. For example, in preparation for bioprinting the cap 160 can be removed from the connector 112 and the nozzle 132 can be connected to the connector 112 to direct the bioink 101 ejected from the barrel 102 during bioprinting. Additionally or alternatively, the nozzle 132 can be a part of (e.g., integrally formed with) the distal end 108.

The adapter 200 can include a needle 202. The needle 202 can be configured to pierce both the stopper 114 and the filter 122. The needle 202 can be sterilized before piercing the stopper 114 and the filter 122 to preserve sterility within the barrel 102. The filter 122 can prevent particulates created when the stopper 114 is pierced from passing distally beyond the filter 122 to preserve sterility. The needle 202 can pierce an exposed portion of the stopper 114, such as the central portion 117. The needle 202 can be connected to a gas supply 204 that can connect to the bioprinter 300. The gas supply 204 can supply gas from bioprinter 300 to the interior of the barrel 102 via the needle 202. The gas supplied from the bioprinter 300 can pneumatically drive the piston 118 distally to eject the bioink 101 from the distal end 108 of the barrel 102. In embodiments, the needle 202 can be integral with the adapter 200. In embodiments, the needle 202 can be separate from the adapter 200 and can be attached to the adapter 200 via a snap fit, screw, push fit, bayonet fit, or other connection.

The needle 202 can be dimensioned such that when connected to the adapter 200 and when the adapter 200 is connected to the syringe 100 the needle pierces both the stopper 114 and the filter 122 but does not contact the piston 118. That is, the adapter 200 can support the needle 202 at a position in which a distal tip of the needle 202 is within the barrel 102 distal to the filter 122 and proximal to the piston 118 when the syringe 100 is filled with the bioink 101. For example, the needle 202 can have a length in the longitudinal direction of between 1-3 cm. In embodiments, the needle 202 can have a diameter of between 1-5 mm.

The adapter 200 can be attached to the proximal end 104 of the syringe 100 via any number of connection mechanisms including for example by snap fitting, clasping, push fitting, threading, bayonet fitting, etc. For example, in embodiments the adapter 200 can include a first body 206 and a second body 208. The needle 202 can extend through the first body 206. In embodiments, the needle 202 can be fixed to the first body 206 using a connector, adhesive, ultrasonic welding, or other technique. In embodiments, the needle 202 can be formed integrally with the first body 206. The first body 206 can include a first connector 210 and the second body 208 can include a second connector 212 that is complementary to the first connector 210. The first connector 210 and the second connector 212 are configured to connect to join the first body 206 to the second body 208. The first connector 210 and the second connector 212 can be snap fit, claps, push fits, threads, a bayonet fitting, or any number of other connectors.

The adapter 200 can include a fitting to fit the proximal end 104 of the syringe 100 to the adapter 200. The stopper 114 can fit within the fitting and thus container closure integrity of the proximal opening 106 can be maintained when the adapter 200 is connected to the syringe 100. In embodiments such as shown in FIG. 6, the second body 208 can include at least part of the fitting. For example, the second body 208 can include a recess 214 and an opening 216. The proximal end 104 of the syringe 100 can rest in the recess 214 and an entirety of the barrel 102 from the distal end 108 up to the flange 105 can extend through the opening 216. According to this configuration and as shown in FIG. 6, the second body 208 can be slid onto the barrel 102 from the distal end 108 to the proximal end 104 and the first body 206 and the second body 208 can sandwich the proximal end 104 within the fitting. The first body 206 and the second body 208 can be configured to align the needle 202 and the stopper 114 such that the needle 202 pierces through the central portion 117 of the stopper 114 when the first body 206 and the second body 208 are connected. For example, the opening 216, the recess 214, the second connector 212, and/or the first connector 210 can together or individually align the needle 202 and the stopper 114 such that the needle 202 pierces through the central portion 117 of the stopper 114 when the first body 206 and the second body 208 are connected. The adapter 200 can comprise a polymer, glass, or other materials. For example, in embodiments the adapter 200 can comprise a cyclic olefin polymer, a cyclic olefin copolymer, and/or a polypropylene.

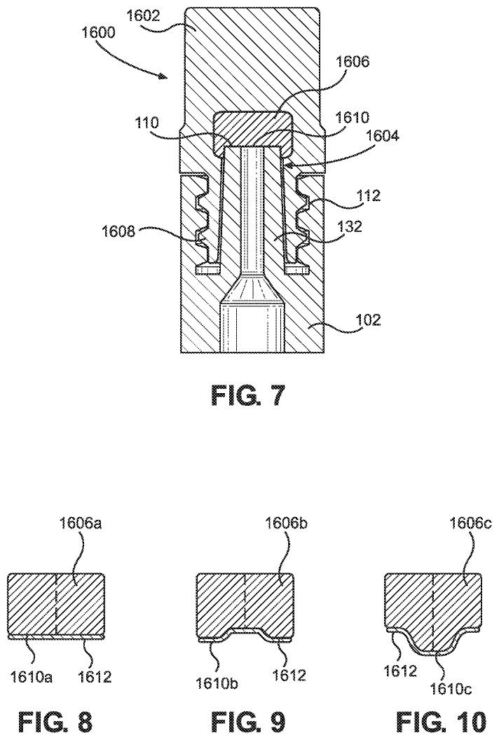

FIG. 7 shows a cross section view of a first example of the cap 1600 removably connected to the barrel 102. The cap 1600 can include any of the features, structures, relationships, etc. of the cap 160, and vice versa. For example, the cap 1600 can be removably connected to the connector 112 of the barrel 102 to seal the barrel 102, as previously described. The cap 1600 can provide container closure integrity (CCI) for the syringe 100 including within low temperature environments.

In embodiments, the nozzle 132 can be an integral part of the barrel 102 and can define the distal opening 110. In embodiments, the nozzle 132 can be a Luer cone tip, though other nozzle configurations are possible. The nozzle 132 and/or barrel 102 can be formed of a number of materials, including for example polymers, such as a cyclic olefin polymer or a cyclic olefin copolymer, and glass, though other materials are possible. In embodiments, the connector 112 can include a Luer lock connection. For example, the connector 112 can include a female Luer lock connection, though other connection types are possible. The nozzle 132 can extend through the connector 112.

The cap 1600 can include a body 1602 that can define a recess 1604. The recess 1604 of the cap 1600 can accommodate the nozzle 132. The cap 1600 can include a seal 1606. In embodiments, the seal 1606 can line an entirety of the recess 1604. In embodiments, the seal 1606 can line the distal end of the recess 1604. The cap 1600 can include a connector 1608. The connector 1608 can be complementary to the connector 112 of the syringe 100 such that the connector 1608 can removably connect to the connector 112. In embodiments, the connector 1608 can be a Luer lock connection. For example, the connector 1608 can be a male Luer lock connection that can be screwed into the female Luer lock connection of the connector 112 to form a connection with the connector 112. Other connection types are possible. For example, in embodiments the connector 1608 can be push-fit and/or snap-fit connection that can be pressed into the connector 112, among other possibilities.

The seal 1606, can engage with the nozzle 132 when the connector 1608 of the cap 1600 is connected with the connector 112 of the syringe 100. In embodiments, this engagement between the seal 1606 and the nozzle 132 can seal the distal opening 110 to provide CCI for the distal end 108 of the barrel 102. The seal 1606 can provide a liquid-tight and/or air-tight seal over the distal opening 110 of the nozzle 132 when the connector 1608 of the cap 1600 is connected with the connector 112 of the syringe 100. The liquid-tight and/or air-tight seal provided by the seal 1606 can preserve CCI at the distal end 108 of the barrel 102 at low temperatures. For example, the liquid-tight and/or air-tight seal provided by the seal 1606 can preserve CCI at the distal end 108 of the barrel 102 at temperatures below −20 °C, including at temperatures below −80 °C, −180 °C, −196 °C, and any temperatures therebetween. The term “low temperature(s)” used throughout this disclosure can include temperatures below −20 °C, including at temperatures below −80 °C, −180 °C, −196 °C, any temperatures therebetween, and/or at any of the previously described cryogenic storage temperatures. Additionally, the liquid-tight and/or air-tight seal provided by the seal 1606 can preserve CCI at the distal end 108 of the barrel 102 at room temperature (e.g., between 20-22 °C), at temperatures between the low temperatures of this disclosure and room temperature, and at temperatures exceeding room temperature.

In embodiments, the seal 1606 can be within the recess 1604 between the body 1602 and a distal and of the nozzle 132. In some such configurations, when the connector 1608 of the cap 1600 is connected to the connector 112 of the barrel 102 the body 1602 and the distal end of the nozzle 132 can impart a compressive force on the seal 1606 so that the seal 1606 can provide the liquid-tight and/or air-tight seal and CCI for the distal end 108 of the barrel 102.

In embodiments, the seal 1606 can comprise an elastomer material, though other materials can be used. The seal 1606 can be mechanically and/or chemically attached to the body 1602 such that the seal 1606 does not detach during thermal expansion/contraction. The seal 1606 can be molded into the body 1602. For example, the seal 1606 can be injection molded into the body 1602 in a two-shot process. In embodiments, the seal 1606 can be over molded onto the body 1602. In embodiments, the seal 1606 can be manufactured separately and pressed into engagement with the body 1602. The seal 1606 can define a number of different shapes and/or sizes.

The seal 1606 can include a proximal face 1610 that can cover and/or seal the distal opening 110 of the nozzle 132 when the connector 1608 of the cap 1600 is connected with the connector 112 of the syringe 100. For example, the seal 1606 can conform to the nozzle 132 or occlude the distal opening 110 of the nozzle 132 at room temperature and/or at low temperatures when the connector 1608 of the cap 1600 is connected to the connector 112 of the barrel 102. The seal 1606 can engage with (e.g., seal) the distal opening 110 of the nozzle 132 and/or exterior walls of the nozzle 132.

FIGS. 8-10 show cross section views of seals 1606a, 1606b, 1606c according to some aspects of the invention. The seals 1606a, 1606b, 1606c can be used with any of the caps 160, 1600, 2600, 3600, 4600, 5600. In embodiments, the proximal face 1610 can be covered with an inert film 1612, which can prevent direct exposure between a substance (e.g., medicament or vaccine) contained within the barrel 102 and the material forming the seal 1606. The inert film 1612 can thus reduce or prevent the material forming the seal 1606 from contaminating the substance contained within the barrel 102. The inert film 1612 can comprise a fluorinated polymer material, though other materials are possible. In embodiments, the seal 1606 can have a substantially flat and/or planar proximal face 1610a, a concave proximal face 1610b, or a convex proximal face 1610c, though other shapes are possible. Different shaped proximal faces can be better suited to provide CCI at different temperatures.

In embodiments, materials of the cap 1600, as well as other embodiments of the cap 160, 2600, 3600, 4600, 5600, can be selected for compatibility with the syringe 100 to ensure CCI at low temperatures. For example, materials that form the cap 160, 1600, 2600, 3600, 4600, 5600 can be selected based on material properties, such as linear coefficients of linear thermal expansion (CLTE). The material properties of the material that forms the cap 160, 1600, 2600, 3600, 4600, 5600 can be compatible with material properties, such as CLTE, of the materials that form the syringe 100. As discussed previously, the syringe 100 can be formed of a number of different materials including for example cyclic olefin polymer, cyclic olefin copolymer, among other possibilities. Because the materials properties of the cap 160, 1600, 2600, 3600, 4600, 5600 can be selected for compatibility with material properties that form the syringe 100, the cap 160, 1600, 2600, 3600, 4600, 5600 and the syringe 100 can change (e.g., contract and/or expand) as a function of temperature at similar rates. This can prevent gaps, cracks, or other artifacts from forming and can provide CCI for the distal end 108 of the barrel 102 in a wide range of different temperatures including for example at the low temperatures described throughout this disclosure.

For example, in embodiments such as the cap 1600 shown in detail in FIG. 7, the body 1602 and the connector 1608 can each be formed of the same material that forms the distal end 108 of the barrel 102, which can include the connector 112 and/or the nozzle 132. Since the body 1602 and the connector 1608 can be formed of the same material that forms the distal end 108 of the barrel 102, the body 1602 and the connector 1608 can have the same material properties as the distal end 108 of the barrel 102. Since the body 1602 and the connector 1608 can have the same material properties at the distal end 108 of the barrel 102 the body 1602, the connector 1608, and the distal end 108 of the barrel 102 can behave similarly (e.g., expand and/or contract) at a number of different temperatures including at the low temperatures described throughout this disclosure, which can help preserve CCI.

In embodiments, the body 1602 and the connector 1608 can be formed of any of the materials discussed above in reference to the barrel 102 including for example polymers such as cyclic olefin polymer, cyclic olefin copolymer, among other possibilities. As would be readily understood to those of skill in the art, same material in this context includes substantial amounts of the same materials such that the cap 1600 and barrel 102 behave similarly (e.g., expand or contact) as a function of temperature. Nevertheless, impurities or composite materials including substantial percentages of the same material can be used provided that the cap 1600 and barrel 102 behave similarly as a function of temperature.

In embodiments, the body 1602 and the connector 1608 can be integral and/or unitary. In embodiments, the connector 112 and the nozzle 132 can be integral and/or unitary.

In embodiments, the material of the seal 1606 can be selected for compatibility with the material that forms the distal end 108 of the barrel 102 including the material that forms the nozzle 132. For example, in embodiments the seal 1606 can be formed of an elastomer. The elastomer forming the seal 1606 can have a CLTE that is greater than and/or within 10% of the CLTE of the material that forms the distal end 108 of the barrel 102 including the material that forms the nozzle 132. For example, the material forming the barrel 102 including the nozzle 132 can include a polymer, such as cyclic olefin polymer, cyclic olefin copolymer, among other possibilities. The polymer can have a CLTE of between 60 and 80 (10⁻⁶/°C), between 65 and 75 (10⁻⁶/°C), between 68 and 72 (10⁻⁶/°C), among other possibilities. The elastomer forming the seal 1606 can have a CLTE that is greater than the CLTE of the material forming the barrel 102. As a result, the seal 1606 can contract faster than the distal end 108 of the barrel 102 and can tighten around the nozzle 132 to maintain CCI at the low temperatures describe in this disclosure.

In embodiments, the material forming the seal 1606 (e.g., an elastomer) can have a glass transition temperature equal to or lower than a temperature that the syringe 100 can be stored at. For example, the material forming the seal 1606 can have a glass transition temperature equal to or lower than any of the low temperatures of this disclosure including for example −80 °C. The seal 1606 can have a CLTE at temperatures below the glass transition temperature of the seal 1606 that is substantially equivalent to the CLTE of the material (e.g., of cyclic olefin polymer) forming the distal end 108 of the barrel 102.

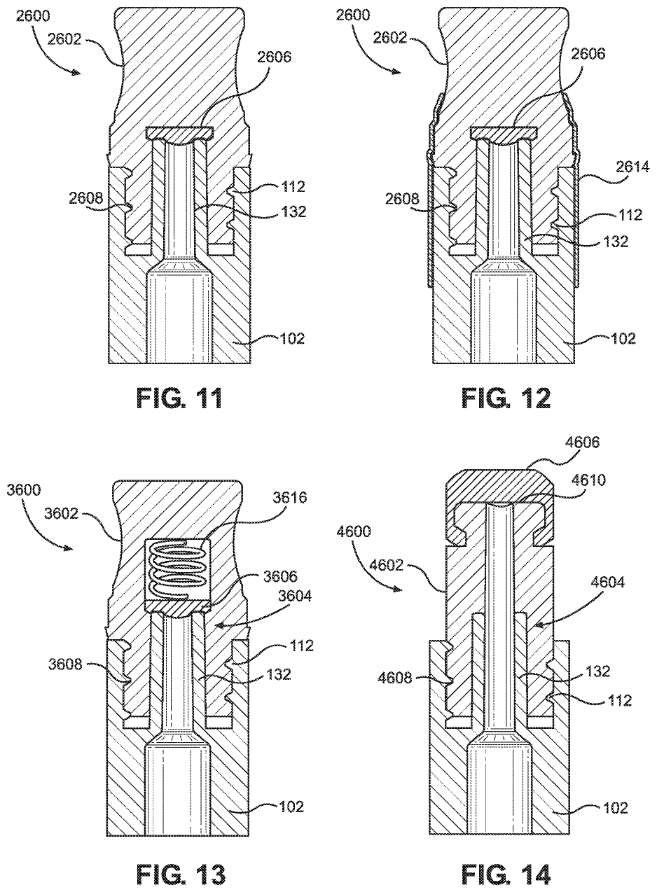

Material selection of the cap 1600 for compatibility with the syringe 100 to ensure CCI at the low temperatures described throughout this disclosure is not limited to selecting the same materials for the cap 1600 and the syringe 100. For example, FIGS. 11 and 12 shows cross section views of a second example of the cap 2600 removably connected to the barrel 102. The cap 2600 can include a body 2602 and a connector 2608 formed of a different material than the material that forms the distal end 108 of the barrel 102. Though the materials of the cap 2600 and the barrel 102 can be different, the materials can be compatible to provide CCI for the distal end 108 of the barrel 102 at low temperatures. In embodiments, material compatibility to provide CCI for the distal end 108 of the barrel 102 can include the material forming the cap 2600 having a CLTE within (+/−) 10% of the CLTE of the material forming the barrel 102. In embodiments, material compatibility to provide CCI for the distal end 108 of the barrel 102 can include the material forming the cap 2600, including the material forming the connector 2608, having a CLTE that is less than the material forming the distal end 108 of the barrel 102, including the material forming the connector 112 and/or the nozzle 132. In embodiments, since the material forming the cap 2600 can have a CLTE that is less than the material forming the distal end 108 of the barrel 102, the connector 112 can contract at a greater rate than the connector 1608 and can grip around the connector 1608 at the low temperatures of this disclosure to provide CCI at the distal end of the barrel.

For example, the material forming the barrel 102 can include a polymer, such as cyclic olefin polymer, cyclic olefin copolymer, among other possibilities. The polymer can have a CLTE of between 60 and 80 (10⁻⁶/°C), between 65 and 75 (10⁻⁶/°C), between 68 and 72 (10⁻⁶/°C), among other possibilities. The material forming the cap 2600, including for example the connector 2608 and the body 2602, can be a rigid plastic material, such as polypropylene. The material forming the cap 2600 can have a CLTE that is the same or different than the CLTE of the material forming the barrel. For example, the material forming the cap 2600 can have a CLTE of between 56 and 76 (10⁻⁶/°C), between 59 and 71 (10⁻⁶/°C), and between 64 and 68 (10⁻⁶/°C), among other possibilities. These material combinations together with the configurations of the connector 2608 and connector 112 and the seal 2606 can preserve CCI at the distal end 108 of the barrel 102 when the connector 2608 of the cap 2600 is connected to the connector 112 of the barrel.

In embodiments such as shown in FIG. 12, when the connector 2608 of the cap 2600 is connected to the connector 112 of the distal end 108 of the barrel 102 the syringe 100 can include a wrap 2614 that is wrapped around the distal end 108 of the barrel 102 and the cap 2600. The wrap 2614 can assist in holding the cap 2600 and the distal end 108 of the barrel 102 together, which can contribute to maintaining a compressive force between the nozzle 132 and the seal 2606 to maintain CCI in a wide range of temperature environments including the low temperature environments of this disclosure. The wrap 2614 can be useful when the materials forming the cap 2600 are different from the materials forming the distal end 108 of the barrel 102. For example, in embodiments when the materials forming the cap 2600 are different from the materials forming the distal end 108 of the barrel 102 the cap 2600 can be designed to include a tolerance (e.g., a gap) at room temperature to accommodate contraction of the cap 2600 and/or the distal end 108 of the barrel 102 when in the low temperature environments of this disclosure. Such a tolerance can help to ensure that the cap 2600 and/or the distal end 108 of the barrel 102 do not break as a result of the contraction that occurs in the low temperature environments of this disclosure. In such embodiments, the wrap 2614 can hold the cap 2600 and the distal end 108 of the barrel 102 together at room temperature to ensure the tolerance does not compromise CCI at room temperature. The wrap 2614 can also be used together with any other cap and syringe 100 embodiments of this disclosure including together with caps 160, 1600, 3600, 4600, and 5600.

Other than differences explicitly described herein, the cap 2600 and seal 2606 can include any features/relationships described in reference to the cap 1600 and seal 1606 where like structures correspond to like structures and vice versa.

FIG. 13 shows cross section views of a third example of the cap 3600 removably connected to the barrel 102. The material that forms a body 3602 and/or a connector 3608 of the cap 3600 can be different from and can have a CLTE that is greater than the CLTE of the material that forms the distal end 108 of the barrel 102 and/or a CLTE that is not within 10% of the CLTE of the material that forms the distal end 108 of the barrel 102. In such embodiments, CCI at the low temperatures can be provided due to structural relationships between the cap 3600 and the distal end 108 of the barrel 102 and/or due to the material forming the seal 3606. For example, the connector 3608 can be configured to connect with the connector 112 of the distal end 108 of the barrel 102 such that the body 3602 and the nozzle 132 and impart a compressive force on the seal 3606 to seal the nozzle 132 at the low temperatures of this disclosure.

In embodiments and as discussed previously in reference to the seal 1606, the material of the seal 3606 can be selected for compatibility with the material that forms the distal end 108 of the barrel 102 including the material that forms the nozzle 132. For example, in embodiments the seal 3606 can be formed of an elastomer. The elastomer forming the seal 3606 can have a CLTE that is greater than the CLTE of the material that forms the distal end 108 of the barrel 102 including the material that forms the nozzle 132. For example, the material forming the barrel 102 including the nozzle 132 can include a polymer, such as cyclic olefin polymer, cyclic olefin copolymer, among other possibilities. The polymer can have a CLTE of between 60 and 80 (10⁻⁶/°C), between 65 and 75 (10⁻⁶/°C), between 68 and 72 (10⁻⁶/°C), among other possibilities. The elastomer forming the seal 3606 can have a CLTE that is greater than the CLTE of the barrel 102. As a result, the seal 3606 can contract faster than the distal end 108 of the barrel 102 and can tighten around the nozzle 132 to maintain CCI at the low temperatures describe in this disclosure. Alternatively, the material forming the seal 3606 can have a CLTE equal to or less than the CLTE of the distal end 108 of the barrel 102.

In embodiments, the cap 3600 can include a force-imparting structure 3616 disposed distally within the recess 3604 between the body 3602 and the seal 3606 that can impart a compressive force on the seal 3606 when the connector 3608 of the cap 3600 is connected to the connector 112 of the distal and of the barrel 102. The force-imparting structure 3616 can help maintain CCI in low temperature environments in embodiments in which the cap 3600 includes materials that differ from the materials forming the distal end 108 of the barrel 102. In embodiments, the force-imparting structure 3616 can also be provided within the recess of any other caps of this disclosure including caps 160, 1600, 2600, 4600, and 5600. The force-imparting structure 3616 can include a spring, a material (e.g., a metal, water, among other possibilities) with a negative coefficient of linear thermal expansion that expands when cooled, and/or a shape memory material such as for example nitinol. Alternatively, the cap 3600 can be provided without the force-imparting structure 3616.

Other than differences explicitly described herein, the cap 3600 and the seal 3606 can include any features/relationships described in reference to the caps 1600, 2600 and seals 1606, 2606 where like structures correspond to like structures and vice versa.

FIG. 14 shows a cross section view of a fourth example of the cap 4600 removably connected to the barrel 102. A seal 4606 can connect over a body 4602 of a cap 4600 to seal the nozzle 132 of the distal end 4610 of the barrel 102. The seal 4606 can be pressed fit onto the body 4602 or over-molded on to the body 4602. The body 4602 can include a connector 4608 that can connect with the connector 112 of the barrel 102. The body 4602 can include a recess 4604 through which the nozzle 132 can extend. In embodiments, the nozzle 132 can be flush with a distal opening of the recess 4604 when the connector 4608 is connected with the connector 112.

Other than differences explicitly described herein, the cap 4600 and the seal 4606 can include any features/relationships described in reference to the caps 1600, 2600, 3600 and seals 1606, 2606, 3606 where like structures correspond to like structures and vice versa.

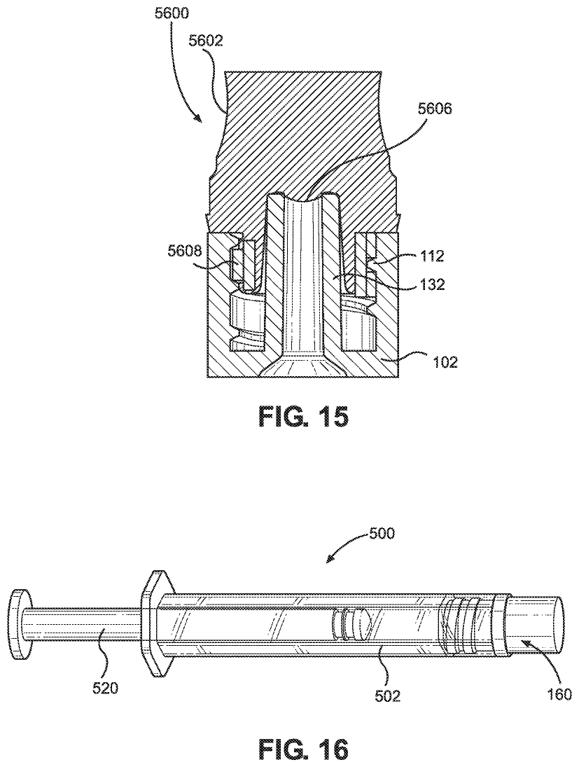

FIG. 15 show a cross section views of a fifth example of the cap 5600 removably connected to the barrel 102. A seal 5606 can be formed integrally with a body 5602 of a cap 5600 using the same material. For example, the body 5602 and the seal 5606 can be molded from an elastomer. In embodiments, a connector 5608 of the 500 can be formed of a material different from the material that forms the body 5602 and the seal 5606. The connector 5608 can be overmolded onto the body 5602. For example, the connector 5608 can be formed of a material that is compatible with the material that forms the distal end 108 of the barrel 102 to preserve CCI at the low temperatures of this disclosure. For example, the connector 5608 can be formed of the same material that forms the distal end 108 of the barrel 102 such as for example a polymer, such as cyclic olefin polymer, cyclic olefin copolymer, among other possibilities. In embodiments, material compatibility to provide CCI for the distal end 108 of the barrel 102 can include forming the connector 5608 out of a different material from the material forming the distal end 108 of the barrel 102. For example, the connector 5608 can be formed of a compatible different material having a CLTE within (+/−) 10% of the CLTE of the material forming the barrel 102. In embodiments, the connector 5608 can be formed of a compatible different material having a CLTE that is less than the material forming the distal end 108 of the barrel 102, including the material forming the connector 112 and/or the nozzle 132.

Other than differences explicitly described herein, the cap 5600 and seal 5606 can include any features/relationships described in reference to the caps 1600, 2600, 3600, 4600 and seals 1606, 2606, 3606, 4606 where like structures correspond to like structures and vice versa.

Any of the caps 160, 2600, 3600, 4600, and 5600 of can be removably connected to other syringes. FIG. 16 shows another syringe 500 having a barrel 502 and a plunger 520. Any of the caps 160, 2600, 3600, 4600, and 5600 of can be removably connected to a distal end of the barrel 502 of the other syringe 500.

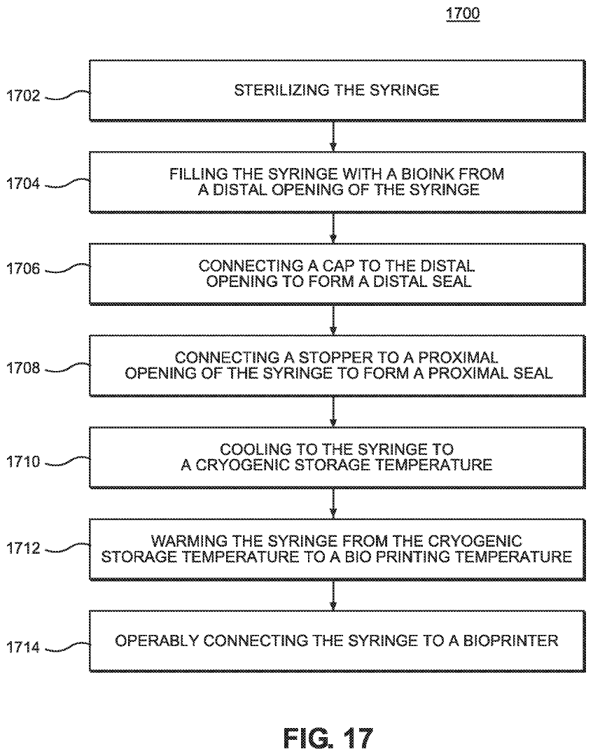

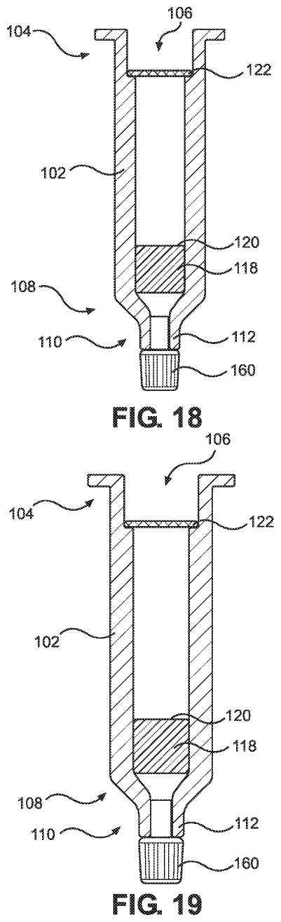

FIG. 17 shows a process 1700 of preparing any of the disclosed embodiments of the syringe 100 for bioprinting. FIGS. 18-25 show the syringe 100 progressing through the process 1700. As shown in FIG. 18, the syringe 100 can be provided in an unfilled and unsterilized state.

As shown in FIG. 19, the process 1700 can include at step 1702 sterilizing the syringe 100. The syringe 100 can be sterilized using any number of sterilization techniques including for example, gamma irradiation, steam autoclave, ethylene oxide, supercritical CO₂, etc. At least regions within the syringe 100 (e.g., the piston 118, the inner wall 103, etc.) that can directly contact the bioink 101 can be sterilized. In embodiments, an entirety of the syringe 100 can be sterilized.

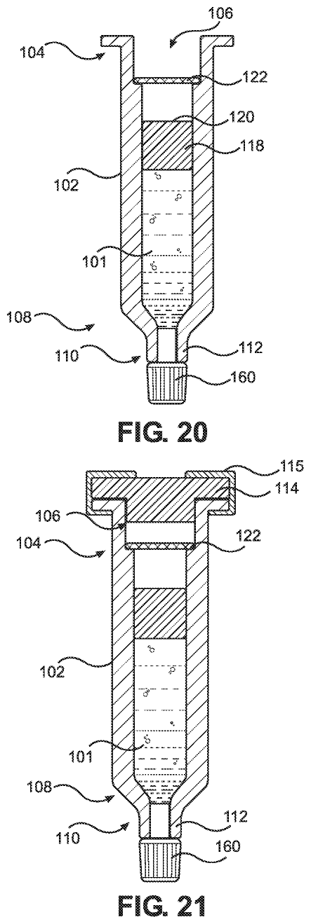

As shown in FIG. 20, the process 1700 can include at step 1704 filling the syringe 100 with a bioink 101. The syringe 100 can be filled from the distal opening 110, which can move the piston 118 proximally as the syringe 100 is filled. The syringe 100 can be filled with a volume of the bioink 101. The volume of the bioink 101 can be based upon a distance that the proximal surface 120 of the piston 118 is spaced proximally from the filter 122 when the syringe 100 is filled. As discussed above, this distance can be associated with a volume of the bioink 101, a material property of the bioink 101, and the range of temperatures the syringe 100 is configured to maintain container closure integrity across. For example, when the barrel 102 is filled with a volume of the bioink 101 and the syringe 100 is configured to maintain container closure integrity at cryogenic storage temperatures (e.g., temperatures below −130 °C, between −196 °C and −130 °C, below −196 °C), the distance can be greater than an amount of linear expansion (i.e., along the longitudinal axis of syringe) of the bioink 101 with the barrel 102 that results from cooling the bioink 101 from a filling temperature to the cryogenic temperature. Since the distance is greater than the amount of linear expansion of the bioink 101, the bioink 101 can freely expand within the syringe 100 and move the piston 118 proximally towards the filter 122 without touching the filter 122 and thus without compromising the functionality of the filter 122. The filter 122 can allow gas (e.g., air) within the barrel 102 to flow through as the piston 118 moves proximally.

As is also shown in FIG. 20, the process 1700 can include at step 1706 connecting the cap 160 to the distal opening 110, e.g., to the connector 112, to form a distal seal.

As is shown in FIG. 21, the process 1700 can include at step 1708 connecting the stopper 114 to the proximal opening 106. The stopper 114 can be connected to the proximal opening 106 via the crimp 115, or any of the other connection techniques described above.

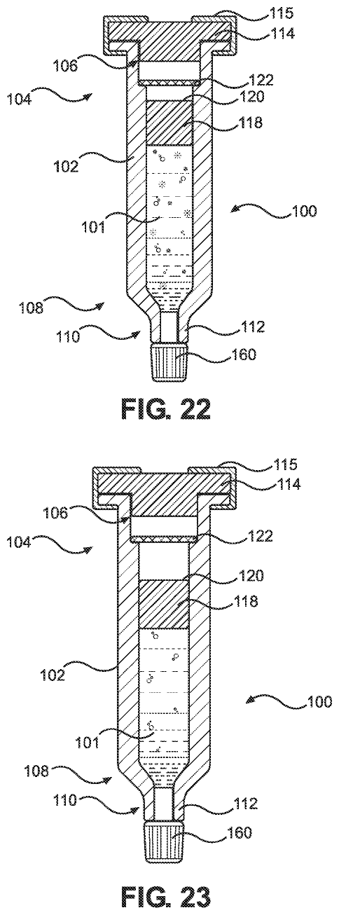

As shown in FIG. 22, the process 1700 can include at step 1710 cooling the syringe 100 to a cryogenic storage temperature. As discussed above, the cryogenic storage temperature can be temperatures below −130 °C, between −196 °C and −130 °C, or below −196 °C. When cooled, the bioink 101 can expand. Since the distance between the filter 122 and the piston 118 was configured to accommodate the expansion of the bioink 101 the bioink 101 can push the piston 118 proximally without contacting the filter 122. Container closure integrity of the seals of the proximal opening 106 and the distal opening 110 can be maintained since the syringe 100 accommodates expansion of the bioink 101 without placing undue stress on the cap 160 and/or the stopper 114. To achieve the cooling, the syringe 100 can be placed in a freezer. Additionally or alternatively, the syringe 100 can also be placed in a secondary container such as a foil bag, tub, or racks for freezing in a liquid nitrogen environment.

As shown in FIG. 23, the process 1700 can include at step 1712 warming the syringe 100 from the cryogenic storage temperature to a bio printing temperature. The bio printing temperature can be a temperature between 0 °C and 40 °C including for example room temperature (i.e., between 20-22 °C). The syringe 100 can be warmed using any number of techniques including dry thawing or by placing the syringe 100 in a water bath. As shown in FIG. 23, the bioink 101 can contract when warmed and in response the piston 118 can move distally towards the distal opening 110. The cap 160 and the stopper 114 can maintain container closure integrity during the warming from the cryogenic temperature.

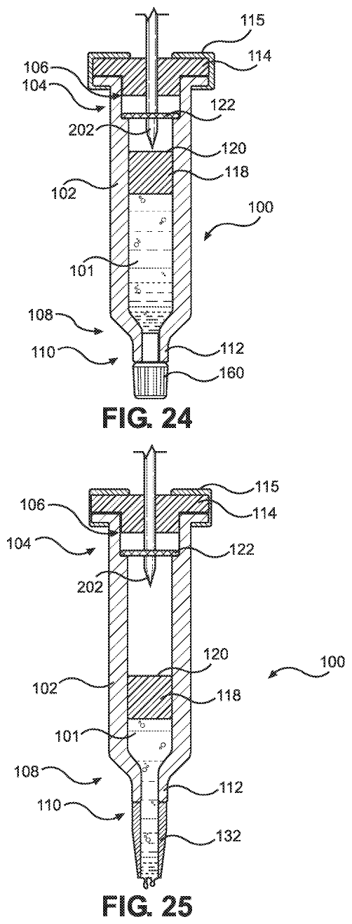

As shown in FIGS. 24 and 25, the process 1700 can include at step 1714 operably connecting the syringe 100 to the bioprinter 300. As discussed above, operably connecting the syringe 100 to the bioprinter 300 can include connecting the syringe 100 to the adapter 200. Step 1714 can occur after step 1712. As discussed above, operably connecting the syringe 100 to the bioprinter 300 can include piercing the stopper 114 and the filter 122 with the needle 202 without contacting the piston 118. To ensure that the needle 202 does not contact the piston 118, the volume of the bioink 101 filled at step 1704 can be based upon a distance that the proximal surface 120 of the piston 118 is spaced proximally from the filter 122 when the syringe 100 is filled and based upon a position of the distal tip of the needle 202 when the needle 202 pierces the stopper 114 and the filter 122. Step 1714 can also include removing the cap 160 and connecting the nozzle 132 to the connector 112 at the distal end 108 of the syringe 100.

The primary container systems and methods of this disclosure can be implemented in other bioprinting and tissue engineering processes beyond pneumatic extrusion, such as mechanical extrusion, FRESH (Freeform Reversible Embedding of Suspended Hydrogel) bioprinting, electrospinning etc. Additionally, the primary container systems and methods of this disclosure can be used in handheld bioprinters for in situ deposition of bioinks and the bioprinters to be used at the point of care. The primary container systems and methods of this disclosure can also be used in cryo-bioprinting processes, where cryoprotectant containing bioink can be printed on an ultra-cold surface for instantly freezing a bioprinted construct.

The primary container systems and methods of this disclosure can also be used for a point-of-care delivery device, such as for allogenic cell therapy product injection (including but not limited to) for osteoarthritis. Standardized allogenic cell therapy for osteoarthritis can be enabled by the primary container systems and methods of this disclosure, where the primary container systems and methods of this disclosure can be used as an off-the-shelf cell product that could be thawed at a clinical site and administered to patients. The Luer-lock end can allow for ready attachment of 22-gauge needle typically used in intra-articular injections.

The primary container systems and methods of this disclosure could also be used for delivering pancreatic islets or similar sized organoid-containing biomaterial formulations subcutaneously or intraperitoneally for treating diabetes and other systemic and genetic diseases. The primary container systems and methods of this disclosure could be an attachment to a syringe injector/gun that can delivery biomaterial drug product subcutaneously or, for example, within an implantable medical device which provides a vascularized environment for therapeutic cells. The primary container systems and methods of this disclosure could also be a laparoscopic delivery device, where the Luer-lock end can be connected to a silicone tubing which can in turn be connected to a trocar and an elongated tube-like assembly for intra-peritoneal delivery into omentum of patients.

The primary container systems and methods of this disclosure can also find application beyond bioprinting, for the pneumatic delivery of cell therapy and other therapeutic materials.

While certain implementations of the invention have been described, the present disclosure is not limited to these disclosed aspects. Additional modifications and improvements to the disclosure may be apparent to those skilled in the art. Moreover, the many features and advantages of the disclosure are apparent from the detailed specification, and thus, it is intended by the appended claims to cover all such features and advantages of the present disclosure which fall within the spirit and scope of the disclosure.

1. A syringe configured to contain a bioink and to provide container closure integrity through cryogenic storage and warming, the syringe comprising:
a barrel comprising a proximal end defining a proximal opening and a distal end defining a distal opening, the distal end comprising a connector;
a stopper at the proximal end, the stopper sealing the proximal opening;
a cap removably connected to the connector, the cap sealing the distal opening; and
a piston within the barrel, the piston is configured to be spaced apart from the stopper when the barrel is filled with the bioink, wherein:
the stopper is configured to seal the proximal opening throughout a temperature range,
the cap is configured to seal the distal opening throughout the temperature range, and
the temperature range is between −196 °C and 37 °C.

2. (canceled)

3. The syringe of claim 1, further comprising a filter within the barrel between the proximal opening and the piston.

4-6. (canceled)

7. The syringe of claim 3, wherein, during cryogenic storge the piston, is spaced distally from the filter.

8-13. (canceled)

14. The syringe of claim 1, wherein a central portion of the stopper is pierceable.

15. The syringe of claim 1, further comprising the bioink within the barrel, wherein the bioink fills the barrel between the piston and the cap.

16. (canceled)

17. The syringe of claim 15, wherein:
the bioink occupies a first volume within the barrel when the syringe and the bioink are at room temperature and the bioink occupies a second volume within the barrel when the syringe and the bioink are at a cryogenic storge temperature, and
the second volume is greater than the first volume.

18. The syringe of claim 1, wherein:
the connector of the distal end of the barrel is a first connector,
the first connector comprises a first material having a first coefficient of linear thermal expansion,
the cap comprises:
a second connector removably connected to the first connector, the second connector comprising a second material having a second coefficient of linear thermal expansion, and
a seal configured engaged with the distal opening of the barrel, and
the second coefficient of linear thermal expansion is less than or equal to the first coefficient of linear thermal expansion.

19. The syringe of claim 18, wherein the second coefficient of linear thermal expansion is within 10% of the first coefficient of linear thermal expansion.

20. (canceled)

21. The syringe of claim 18, wherein the seal conforms to the distal end of the barrel or occludes the distal opening of the barrel at room temperature.

22. The syringe of claim 18, wherein:
the seal comprises a proximal face that is engaged with the distal end of the barrel, and
the cap comprises an inert film that lines the proximal face.

23. The syringe of claim 18, wherein:
the seal comprises an elastomer,
the elastomer of the seal has a third coefficient of linear thermal expansion, and
the third coefficient of linear thermal expansion is within 10% of the first coefficient of linear thermal expansion.

24. (canceled)

25. The syringe of claim 18, wherein the distal end of the barrel comprises the first material,
the seal comprises a third material having a third coefficient of linear thermal expansion, and
the third coefficient of linear thermal expansion is greater than the first coefficient of linear thermal expansion.

26. The syringe of claim 1, wherein:
the cap comprises:
a body defining a recess;
a connector removably connected to the connector of the barrel; and
a seal engaged with the distal opening, and
the seal comprises a first material and the body comprises a second material, and the first material and the second material are different.

27. The syringe of claim 26, wherein:
the seal is within a distal end of the recess,
the seal comprises a proximal face that seals the distal opening of the barrel, and
the proximal face is lined with an inert film.

28. (canceled)

29. The syringe of claim 26, further comprising a spring within the recess between the body and the seal, wherein the spring imparts a compressive force on the seal to seal the distal opening of the barrel.

30. The syringe of claim 26, further comprising a material within the recess between the body and the seal, wherein the material has a negative coefficient of thermal expansion and imparts a compressive force on the seal to seal the distal opening of the barrel when the material is cooled.

31. The syringe of claim 26, wherein the seal has a coefficient of linear thermal expansion at temperatures below a glass transition temperature of the seal that is substantially equivalent to a coefficient of linear thermal expansion of cyclic olefin polymer.

32. The syringe of claim 1, wherein:
the cap comprises:
a connector removably connected to the connector of the distal end of the barrel; and
a body connected to the connector of the cap and engaged with the distal opening of the barrel, and
the connector of the cap comprises a cyclic olefin polymer and the body comprises an elastomer.

33. An assembly for a bioprinter, the assembly comprising:
a syringe comprising:
a barrel comprising a proximal end defining a proximal opening and a distal end defining a distal opening, the distal end comprising a connector;
a stopper at the proximal end, the stopper sealing the proximal opening;
a bioink within the barrel;
a piston within the barrel between the stopper and the bioink; and
a filter within the barrel between stopper and the piston;
an adapter configured to connect to the proximal end of the barrel; and
a needle extending through the adapter and configured to pierce both the stopper and the filter.

34. The assembly of claim 33, wherein the needle is configured to pneumatically supply a gas within the barrel to move the piston distally and dispense the bioink from the distal end of the barrel.

35-76. (canceled)
